(19) United States
(12) Patent Application Publication
(10) Pub. No.: US 2011/0195489 A1
(43) Pub. Date: Aug. 11, 2011

(54) System and Method of an In-vitro Wound Healing Model on Cultured Cell Monolayer Employing Stamp Device

(76) Inventor: Rongpei Lan, San Antonio, TX (US)

(21) Appl. No.: 13/022,349

(22) Filed: Feb. 7, 2011

Related U.S. Application Data
(60) Provisional application No. 61/302,455, filed on Feb. 8, 2010.

(51) Int. Cl. C12M 3/00

(52) U.S. Cl. 435/283.1

(57) ABSTRACT

A system and method of an in vitro wounding model that employs a wounding device compromising a stamp assembly with a stamp disk engraved with alternating grooves and ridges, to dislodge cells of cultured cell monolayer. Strips or islands of cells are left behind, which can be used for morphological observation in biological research. Further and more importantly, these strips or islands of cells can be designed to be sufficiently narrow or small (˜100 μm) to ensure that all remaining cells become rapidly activated and then migrate, dedifferentiate and proliferate in near synchrony, so that samples can be collected in large amount which is suitable for biochemical studies.

### CROSS-REFERENCE TO RELATED APPLICATIONS

This application claims the priority and the privilege within the scope of a provisional patent (No. 61/302,455) filed on Feb 8, 2010.

### STATEMENT REGARDING FEDERALLY SPONSORED RESEARCH OR DEVELOPMENT

This application is supported by National Institutes of Health research program (OK37139/M.A.V), and the inventor has been granted full privilege from University of Texas Health Science Center At San Antonio.

### BACKGROUND OF THE INVENTION

1. Technical Field

The invention relates to a system and method of an in vitro wound healing biological model which employs a stamp device to denude cells of a cultured cell monolayer.

2. Description of the Prior Art

Wounding or cell death in tissues such as skin, intestine, airways and kidney tubules would lead to series of healing responses including migration, proliferation and differentiation of epithelial cells, trying to cover the wounded area. Extensive research work has been studied on these complex events in in-vivo models of wound healing in experimental animals. But in such in vivo studies, the identification and characterization of epithelial specific responses is made difficult by confusion caused by the simultaneous activation of multiple signaling pathways in several cell types.

In vitro wound healing model compromising dislodging part of cultured cell monolayer has been also widely used to mimic the cellular responses in injured tissues or organs in biology field. Such in vitro experiments provide a clean environment where epithelial specific responses can be identified and characterized, which is very important in studying or screening drugs by observing their effect on a specific cell type. Surely, isolation or sampling of cells left behind after wounding in vitro wound healing model is the primary requirement for the relating biochemical studies, in additional to the routine morphological observations.

Prior arts used in vitro wound-healing models employ plastic pipette tips, needle points, scalpels, scrapers, floating pin arrays, or plastic hair combs to dislodge narrow strips of cell monolayer in a culture container. Such techniques yield visible wounds bounded by large areas of intact epithelium. Wounding with electrical current was also tried with an advantage of monitoring cell proliferation by measuring the impedance of culture substrates, but this prior art leaves behind electrically scorched or coagulated cell debris on the culture substratum, which may hamper uniform migration of remaining cells. These prior arts do not permit direct sampling of whole wounded cell monolayer due to low signal to noise ratio regarding cell activation signaling, so they are only suitable for morphological studies such as microscopic observation or immunostaining studies.

Considering only few cells adjacent to wound edges are participating in the wound healing responses in these techniques, several prior arts had been done in order to collect samples of ‘pure activated cells’, to avoid high noise to signal ratios.

Laser capture microdissection has been tried to dissect and collect activated cells from wound edges and unperturbed cells from distant areas. This laborious and time-consuming procedure usually results in only very small size of samples can be obtained, thus normally limiting its use only in studies of nuclear acids such as DNA or RNA which can be enormously amplified readily by Polymerase Chain Reaction technique.

Some prior arts have attempted to produce more extensive wounding on the culture monolayer, in order to let more cells participate the wound healing process. A scraping device was designed to produce a continuous spiral curvilinear wound extending from the center of the culture dish to its periphery. However, even with this improvement, only 40% of the remaining cells were involved in the wound-healing process, and scratches are very apparent on the wounded substratum which would hamper the cell migration. Shark's tooth gel sequencing combs were used to wound cultured cell monolayers along multiple axes, but this prior art still yields inconsistent mixture of large or small cell islands.

These problems existing in all these above-described prior arts have been largely responsible for the infrequent use of wound healing models as paradigms for the biochemical and molecular biological analysis of regenerating epithelium. This type of information in vitro experiments is critically required to be placed in the context of other data from in vivo models of epithelial regeneration for better understanding the epithelium-specific processes during repair and regeneration.

Consequently, there has been a need for an ideal and more efficient in vitro wound healing model which can yield experimental samples sufficient for biochemical studies on regenerating cells. Criterion for an idea intro wound healing model would preferably and mainly include: simple, quick, reproducible, capable to produce tiny cell islands or bands, clear wounding bed, minimal damage to the substratum without creating scratches, easy to be learned, high throughput wounding and sample handling, viable and healthy remaining cells, customizable size of remaining cell strips or islands.

The goal of present invention is to design and validate a new wound healing method using a stamping device, which could fulfill most of the above stated requirements for in vitro wound healing research.

The other advantages of the present invention over prior arts will be apparent to those skilled in the art upon reading the following description.

### SUMMARY OF THE INVENTION

This document discloses a method and apparatus for in vitro wound healing biological model on cultured cell monolayer, by employing varying embodiments of stamp assemblies and wounding devices that control these stamp assemblies. ‘Stamp assembly’ is also referred as ‘stamp’ occasionally for convenience in description.

The general principle of the present invention is that the application of the stamps with concentric or parallel grooves on cultured cell monolayer followed by rotation or parallel movement of stamps would result in reproducibly uniform shearing and removal of cells in desired patterns, leaving behind concentric or parallel cell strips, or even cell islands if two consecutive wounding operations are done at a angle to each other on same cell monolayer. The cells would then migrate, differentiate, proliferate, serving as an in vitro wound healing biological model. When the width of grooves are designed to be small enough, the cell strips or cell islands can also be small enough to ensure nearly synchronized activation, so the cells can be harvested as a sample which is nearly homogenous and suitable for biochemical analysis.

According to a first aspect of the present invention, there is an embodiment of stamp assembly comprising a stamp disk and a support block, with alternating concentric circular grooves and ridges on the stamp disk. Radial grooves are engraved further to allow free flow of fluids and air in between the stamp disk and cell monolayer, to prevent vacuum or suction force while lifting a stamp from the cell monolayer. These radial grooves were found by the applicant to be critical in improving wounding efficacy and performance, and the uniformity of cell strips or islands.

According to a second aspect of the present invention, there is an embodiment of stamp assembly comprising a stamp disk and a support block, with alternating parallel grooves and ridges on the stamp disk. The support block is further divided into an upper part and a lower part, with the stamp disk attached and fixed to the upper part. A means of sliding platform and guiding groove fitting ensures that the upper part with the stamp disk would move over the lower supportive part along the axis of parallel grooves. Rotation of an adjustment bolt would be translated to the linear movement of the sliding platform. One wounding procedure on a cultured cell monolayer would produce parallel cell strips, and a second wounding at right angle to the first wounding on same cultured cell monolayer would finally generate cell islands.

Another embodiment of a device and apparatus comprises: a main stand; a stamp assembly comprising a stamp disk and a support block, and the stamp assembly connected to a shaft of the main stand; a holding device for cell culture container holding the cell culture container under the stamp assembly. The stamp assembly is moved to contact with the cultured cell monolayer in the cell culture container to dislodge cultured cells in designed pattern.

Also described is an embodiment of device to control scraping with a stamp assembly, which comprises a pneumatic actuator, a pressure regulator and a control button. The stamp assembly is mounted at the end of a shaft driven by the pneumatic actuator and can be pushed to or lifted from the cell monolayer in a culture container. The pneumatic actuator controls the moving speed of the stamp assembly, and it also provides controlled pneumatic pressure of the stamp disk on the cell monolayer, therefore it greatly improves stability, reproducibility and less variation between each wounding operations.

Also described is an embodiment of a stamp array assembly which contains multiple stamps. The stamp array assembly can be applied to wound multiple rows of culture wells on a cell culture plate, and then rotated together, such as by a gear belt which is linking all stamps. Such a stamp array is very useful if a lot of wounded cultured cell monolayers are demanded, providing a much quicker and high throughput method of wounding multiple wells on a culture plate simultaneously. The stamps of the stamp array assembly are designed to have an adjustment means, allowing slight adjustment on the extension of stamps from the holding plates, so as to ensure that all stamps in the stamp array can have optional contact with the cell monolayer.

In one embodiment of present invention by employing a stamp engraved with sufficiently small grooves, or by consecutive wounding procedures on same culture monolayer, the present in vitro wounding device also provides a means to denude cultured epithelium in patterns designed to leave behind strips or islands of cells sufficiently narrow or small to ensure that all remaining cells become rapidly activated and then migrate, dedifferentiate and proliferate in near synchrony. Therefore signals specific to regenerating cells do not become diluted by quiescent differentiated cells that are not affected by wound induced activation. The near synchronous regenerative activity of cell bands or islands would permit the collection of samples large enough for biochemical studies to sensitively detect alterations involving mRNA for several early response genes and protein phosphorylation in major signaling pathways.

Though preferably the stamp disk of the stamp assembly is made of rubber, it can be made of any material which is suitable for cultured cells and the substratum surface where they grow on.

### BRIEF DESCRIPTION OF THE DRAWINGS

FIG. 1 is a diagram showing wounding patterns by applying stamps engraved with concentric or parallel grooves and ridges on cultured cell monolayer according to one embodiment of the present invention.

FIG. 2 is a perspective view of a stamp assembly engraved with alternating concentric circular grooves and ridges and comprising a smaller neck according to one embodiment of the present invention.

FIG. 3 is a perspective view of a stamp assembly engraved with alternating concentric circular grooves and ridges according to one embodiment of the present invention.

FIG. 4 is a perspective view and exploded view of a stamp assembly engraved with alternating parallel grooves and ridges and with a means of sliding linear movement according to one embodiment of the present invention.

FIG. 5 is a perspective view and exploded view of a stamping device controlled by hand according to one embodiment of the present invention.

FIG. 6 is a perspective view and exploded view of a stamping device which employs a pneumatic means to apply controlled pressure according to one embodiment of the present invention.

FIG. 7 is a perspective view and exploded view of a stamp assembly which contains an upper part and lower part with an extension adjustment mechanism according to one embodiment of the present invention.

FIG. 8 is a perspective view and exploded view of a stamp array assembly which contains multiple stamp assemblies shown in FIG. 7 according to one embodiment of the present invention.

FIG. 9 is an illustration of part of a stained dish and time-lapse still frames of a cell band after wounding.

FIG. 10 is an illustration of a part of a stained dish and time-lapse still frames of a cell island after wounding.

FIG. 11 is an illustration of the Western Blot protein analysis of a cell sample collected from cell bands after wounding.

### DESCRIPTION OF PREFERRED EMBODIMENTS OF THE INVENTION

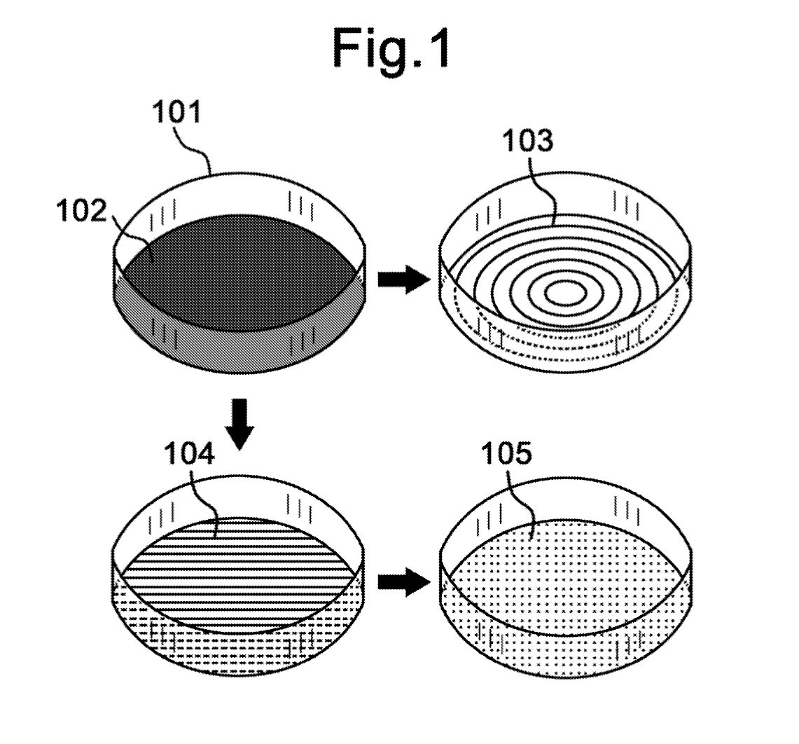

Diagrammatic representation of wounding patterns resulting from rotation of stamps engraved with concentric grooves and ridges, or parallel scraping of parallel stamps with parallel grooves are shown in FIG. 1.

On the inner surface of a cell culture dish 101 or a well 101 of culture plate, cells were grown into a monolayer 102, preferably a layer of confluent and packed cells tightly sticking to each other and to the substratum by cell junctions and other cell anchoring proteins.

In one pattern of cell dislodging method of present invention, a stamp engraved with alternating grooves and ridges in pattern of concentric circles is applied to contact with a cell monolayer 102, and rotation of this stamp would denude cells and leave behind concentric cell strips 103.

In another pattern of cell dislodging method of present invention, a slightly elliptical stamp engraved with alternating grooves and ridges in parallel, linear pattern is applied to contact with a cell monolayer 102, and sliding of this stamp along the axis of ridges would denude cells and leave behind parallel cell strips 104. If cell islands 105 are desired, this parallel stamp is lifted after the first wounding step, rotated 90 degrees, applied on to the same cell monolayer again, and wounding was done once more.

A variety of stamp assemblies have been constructed and designed by the applicant to produce wounding beds of a desired size (200-1000 μm) leaving behind cell bands of a desired width (50-200 μm).

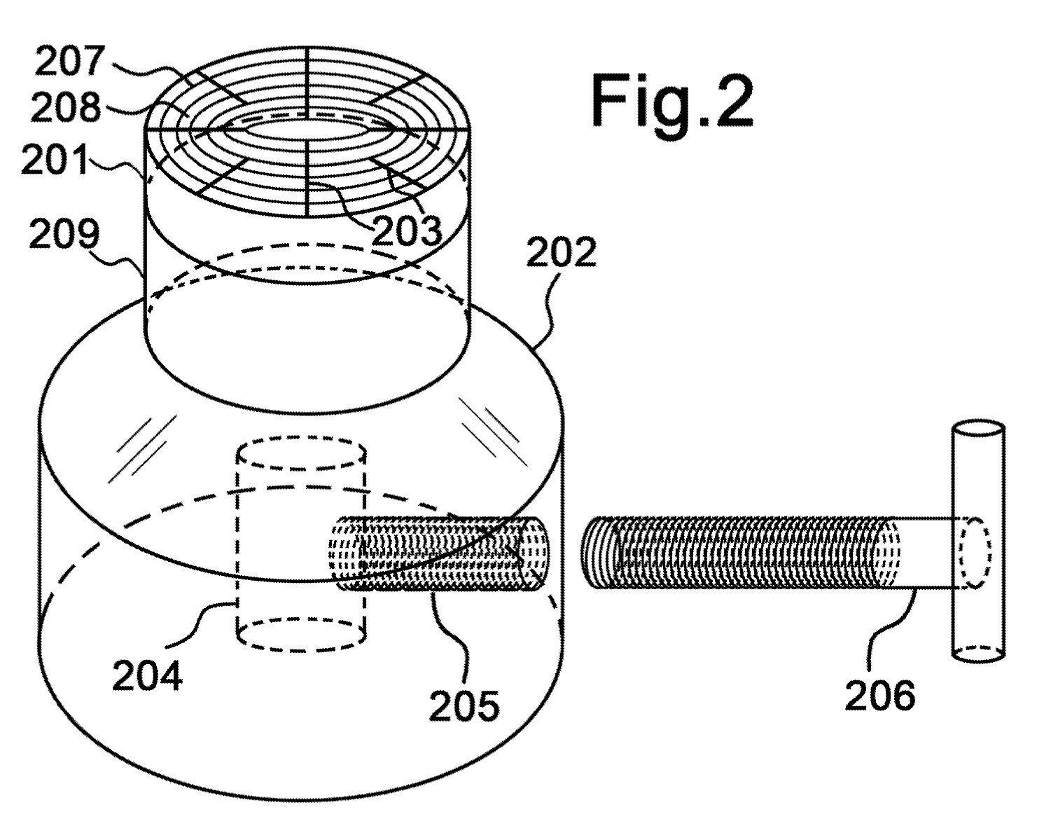

In one embodiment of present invention as shown in FIG. 2, a stamp assembly for small size dishes (35 mm) or wells on a 6-well culture plate is preferably composed of a support block 202 with a small neck 209.

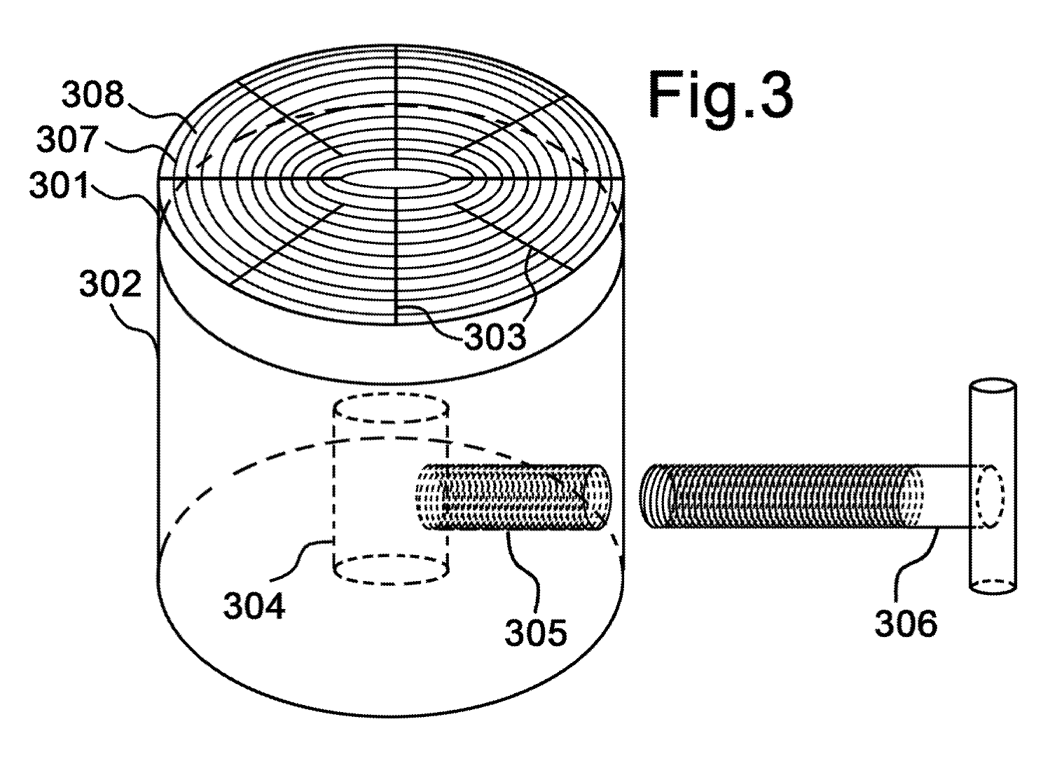

As one embodiment of present invention, a stamp assembly for bigger size dishes (60 mm or 100 mm) without a neck is shown in FIG. 3.

Similarly in FIG. 2 and FIG. 3, a stamp assembly is composed of a flat rubber circular disk 201, 301 is glued and attached tightly to a support block 202, 302. The support block is preferably a cylinder of stainless steel or aluminum alloy, but a plastic material is also can be used instead. The disk is preferably made with rubber, but any material suitable for wounding cells with minimal scratches left behind can be used instead. The support block and the disk is preferably cylinder shape, but some modifications and variations can be also be easily and readily made in present invention by a person with knowledge in the art, following the general principles of the invention.

The stamp disk 201, 301 is engraved with alternating ridges 208, 308 and grooves 207, 307. Radial grooves 203, 303 are further engraved on the rubber disk surface to permit free flow of fluid, especially when the stamp is lifted away from the cell monolayer after wounding, so as to avoid building of hydraulic pressure or vacuum suction on cells which may cause undesired turbulence, unpredictable cell damages and uneven wounding patterns.

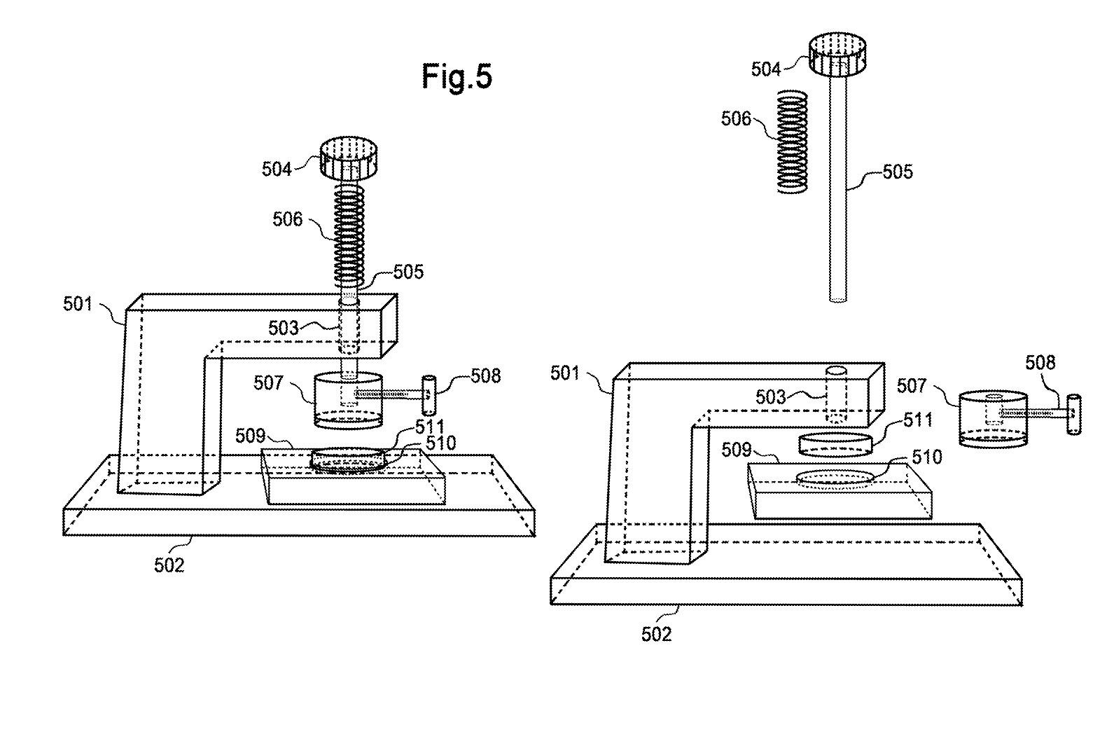

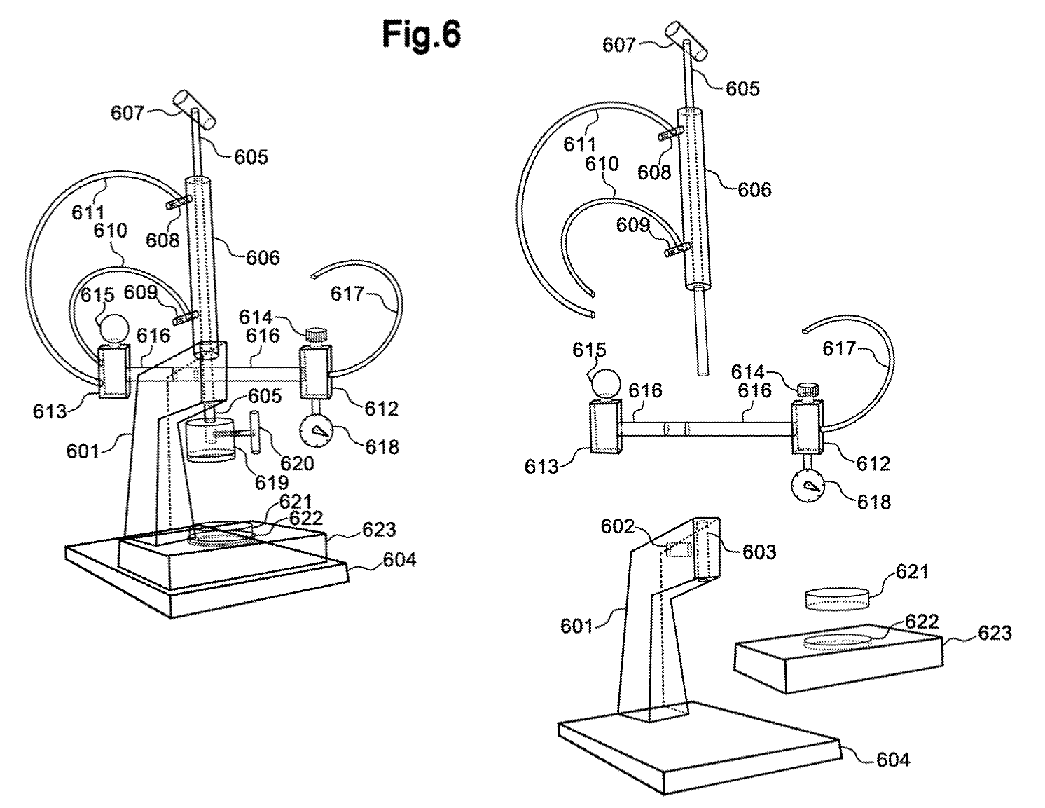

A central bore 204, 304 is created in the stamp support block to fit and locked onto the end of a shaft 505, 605 of a device (in FIG. 5 or FIG. 6) to control the stamp assembly, by a locking bolt 206 306 entering through a threaded bore 205 305 at the side of the support block 202, 302.

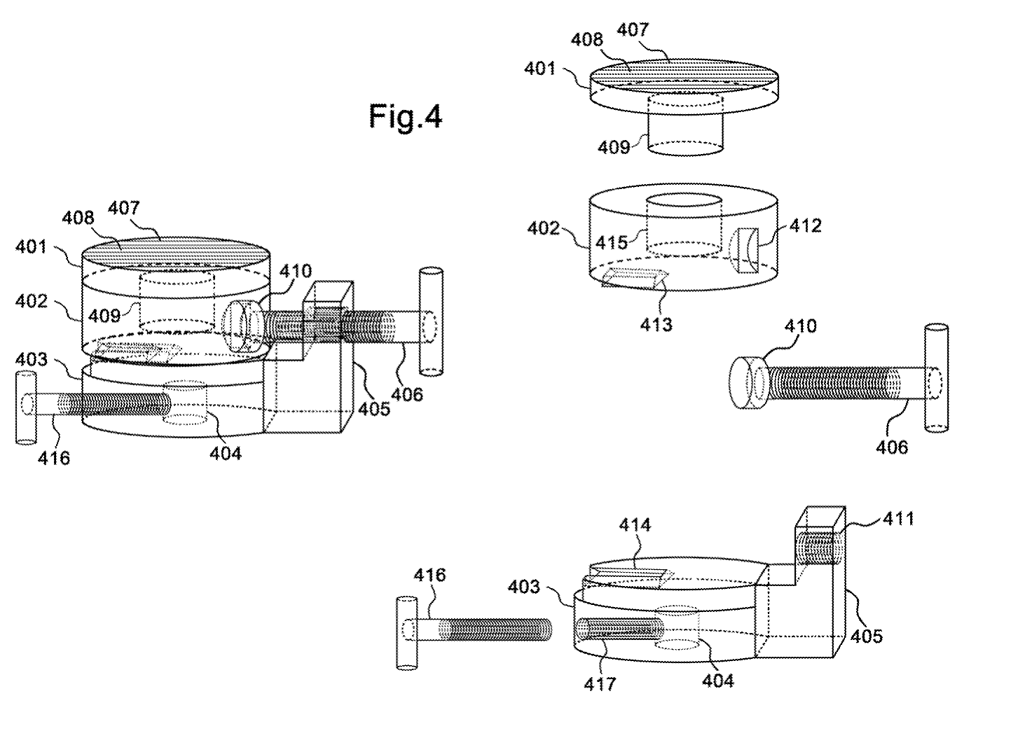

Similar to stamp assembly in FIG. 2 and FIG. 3, as, a further preferred embodiment of a stamp assembly for parallel movement (parallel stamp), as illustrated in FIG. 4, comprises a rubber disk 401 engraved alternating parallel ridges 408 and grooves 407. This rubber disk 401 is mounted on a sliding platform, as the support block, which comprises of an upper supportive part 402 and a lower supportive part 403. The upper supportive part 402 can move along the parallel axis of the engraved ridges 408. In a preferred embodiment of present invention, the upper supportive part 402 connects to and slides on the lower supportive part 403 by means of a fitting block 413 of the upper supportive part perfectly residing in a sliding groove 414 of the lower supportive part guiding the parallel movement of the supportive part 402. An adjustment bolt 406 goes through a threaded bore 411 on a hub 405 at the side of lower supportive part 403, with its round bigger-sized cylinder head 410 perfectly fitting into a cache 412 of the upper supportive part 402. Rotation of the adjustment bolt 406 would be translated to the linear movement of the sliding platform along the axis of the parallel grooves 407. Similar to the other stamp assemblies in FIG. 2 or FIG. 3, a central bore 404 is for connecting to the end of a shaft 505, 605 of a stamping device in FIG. 5 or FIG. 6, which can be locked by a locking bolt 416 going through a threaded bore 417 on side of the lower supportive part 403. A commercial precision sliding platform can be readily used instead to improve performance. Parallel stamp is preferably slightly elliptical in configuration to allow at least 0.3-0.7 cm sliding movements within the confines of circular culture dishes. But this sliding platform is not necessarily limited to such a guiding groove means, any sliding mechanism to move the stamp disk along the axis of the parallel grooves can be readily incorporated in present invention to design a parallel stamp assembly, which should be included in the scope of present invention.

In an embodiment of present invention, a simple device assembly for operating stamps is illustrated in FIG. 5. The stamp assembly is mounted at the end of a shaft 505 which is going through a snug-fitting vertical bored tunnel 503 on a main stand 501, and locked with a locking bolt 508. By pulling, pushing or holding with a hand on the handle 504 at the top of the shaft 505, with assistance of a spring 506 mounted to the shaft 505, the stamp could be moved vertically up, down or holding on the cultured cell monolayer in the cell culture dish 511 held in an engraved cache 510 of the holding plate 509. Then the stamp can also be rotated to dislodge the cultured cells. A stamp assembly of circular or parallel pattern shown in FIG. 2, FIG. 3 or FIG. 4 can be mounted and operated with this simple stamping device.

Yet in another embodiment of present invention as illustrated in FIG. 6, a stamping device assembly for operating stamps can be controlled by a pneumatic means. Modified from the simple device in FIG. 5, a pneumatic actuator 606 is mounted to and drives the shaft 605. The air flow comes from a tubing 617 connecting to either a house compressed air system or a compressed air tank. A pressure regulator 612 with hand knob 614 is included in the pneumatic pathway to provide a controllable pressure. The pressure is indicated by a pressure gauge 618 with measurable range which the applicant recommends to be up to 250 psi. Two miniature flow controllers 608, 609 for the actuator 606 connects to a multi-way air valve 613 with a control button 615, by tubings 610, 611. One miniature flow controller 608 adjusts the speed of lowering the stamp, while the other one 609 adjusts the speed of lifting the stamp. When operating, a stamp 619 is mounted and locked by a locking bolt 620 at the end of the shaft 605, and a culture dish 621 is placed and held tightly in an engraved cache 622 in a holding plate 623. The control button 615 is used to open or release the air flow, which would drive the actuator 606 to move the stamp 619 down to contact with the cell monolayer or lift the stamp 619 from the dish 621. When the stamp 619 is pressed against and in good contact with the cell monolayer, the stamp 619 can be moved circularly by a hand. A stamp assembly of circular or parallel pattern shown in FIG. 2, FIG. 3 or FIG. 4 can be mounted and operated with this stamping device. For parallel stamps and stamp array assembly, this pneumatic assembly is preferred to facilitate operation. By trial and error, we optimized the pressure to be ˜40-50 psi for 35 mm/60 mm Petri dishes, and ˜140 psi for 100 mm Petri dishes.

Similarly, a modified embodiment can readily be developed by using electrical means comprising an electrical actuator, an electrical pressure regulator, and an electrical control button to replace these corresponding parts in the embodiment with pneumatic means.

Although an engraved cache is shown as an example of present invention to hold a cell culture container such as a culture dish or a culture plate, any holding mechanism can be readily employed in present invention.

For the concentric stamps in present invention, by holding a locking bolt with hand, the stamp is manually rotated through an arc of 30-60 degrees to shear the cells away from the culture substratum, thus wounding the monolayer in a concentric pattern. The arc of rotation was empirically determined for a given experiment by examination of the wounding pattern, the widths of wounds and surviving cell bands. Due to imperceptible variations of width in ridges on the stamp, increasing the arcs of rotation tended to make the wounds wider and therefore, the cell bands became narrower. For this reason the applicant found it advisable not to increase the arc of rotation beyond 60 degrees.

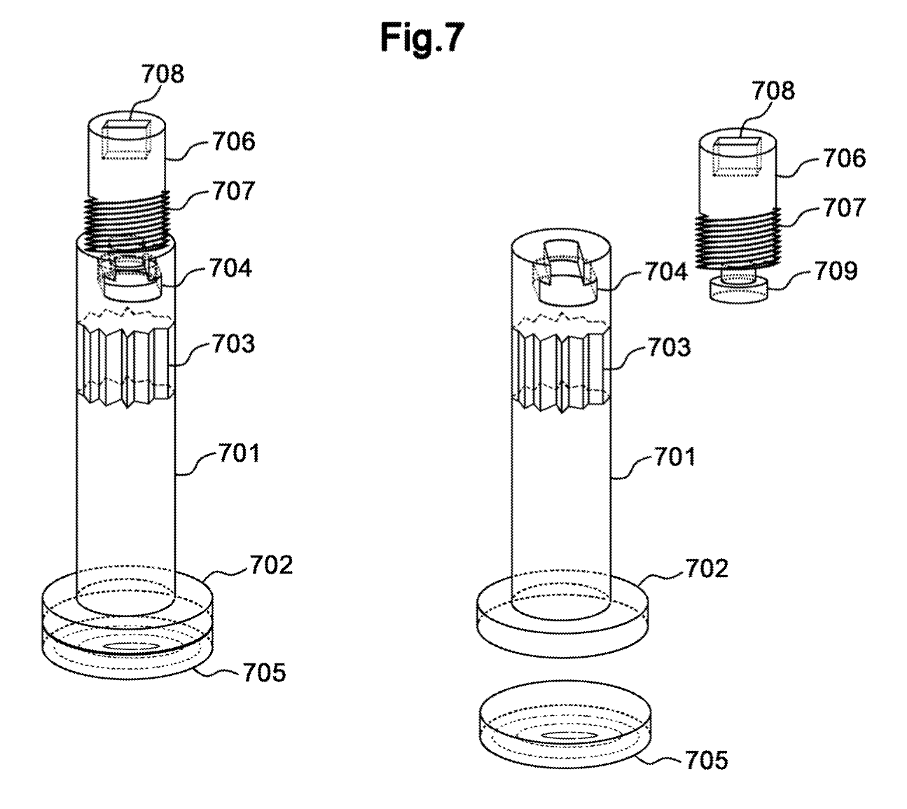

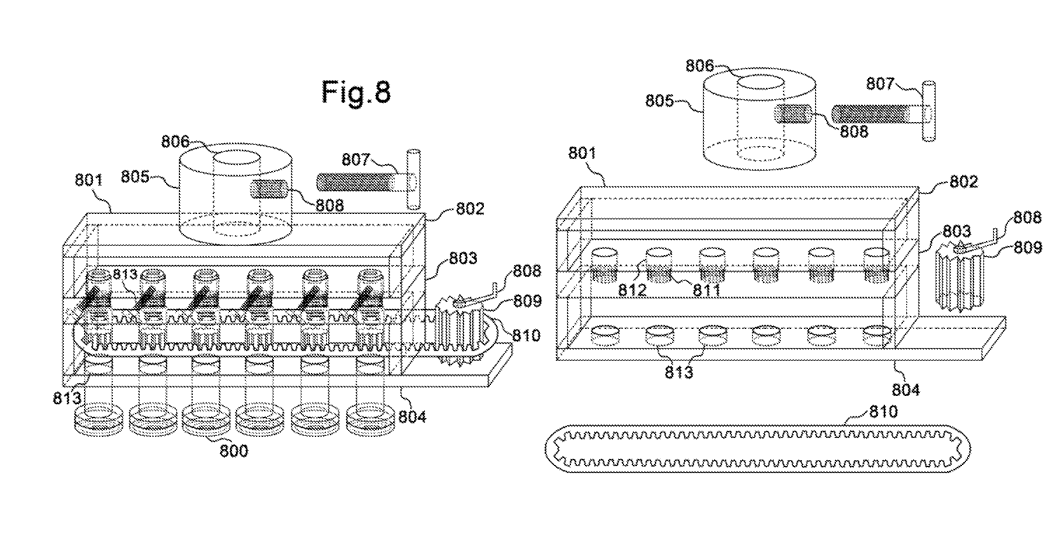

Yet in another embodiment of present invention as illustrated in FIG. 7 and FIG. 8, a stamp assembly is designed to have the capability to adjust its extension, and it can be linked together to form an array of stamps. This stamp array can be applied to quickly wound the wells on a culture plate which contains multiple lines of wells. For this purpose, though not shown in the pictures, it can be readily understood that the holding device 509 or 623 on the wounding control device in FIG. 5 or FIG. 6 should be designed to fit and hold the multi-well culture plate.

As shown in FIG. 7, the stamp comprises of a head part and a main part with a stamp disk 705 mounted at the end. The main part also comprises a geared part 703 for the gear belt 810.

The stamp array assembly in FIG. 8 comprises three layers of plates as the holding device 801, and stamps in FIG. 7 are anchoring on both middle plate 803 and lowest plate 804. A means is employed in this embodiment of present invention to allow slight adjustment on the extension of the stamp disk from the lowest plate so as to achieve optimal contact of the whole array of stamps on the culture plate containing multiple culture wells. This slightly adjustment can be performed as alignment before applying the stamp array to the culture plate, but also even when the stamp array is already contacting the culture plate, so as to achieve least variation between stamps in the array when scraping. The middle plate 803 comprises multiple bores. Each bore 803 further comprise threads 811 on part of the tunnel surfaces while the remaining part 812 is without threads, corresponding to and holding the upper heads of the stamps. Correspondingly, the head part of the stamp also comprises partial surface with threads 707 with remaining smooth tunnel 706. A small T-tail 709 of the head part fits a corresponding T-groove 704 of the main part of stamp, so this connection means would hold the main part but still allow the main part to rotate when the head part is fixed and locked by the small bolt 813 on the middle plate 803 by going through threaded bores at the side of middle plate 803. The stamps further go through and anchor on non-threaded bores 813 on lowest plate 804.

The stamp array is linked and can be rotated together with a gear belt 810. This gear belt is controlled by a drive gear 809 with a hand knob 808. When using, a head connector 805 on the upper plate 802 is mounted to main shaft 605 of the pneumatic device through a central bore 806. This connection can be locked by a locking bolt 807 through threaded bore 808 at the side.

The head part can be locked with a small bolt 813 entering from side of middle plate 803. When lock bolt 813 is released, a small adjustment bar can enter and fit into the adjustment hole 708 on the end of head part, and rotate the head part, so as to adjust the extension of the stamp 800 from the lowest plate 804, for a better alignment of the whole array of stamps. The stamping device in FIG. 6 driven by a pneumatic piston is preferred to operate such a stamp array assembly in FIG. 8. Although a gear belt is shown as an example to link and move the array of stamps together, some modified means can be used readily to do such a work. And although only 1 row of stamps array is described as an example in this document, multiple rows of stamps can be connected and moved by similar mechanism.

Immediately before applying to a cell monolayer, per requirements in cell culture work, the stamp is quickly sterilized with 75% alcohol, rinsed with phosphate buffered saline (PBS) and then primed with culture medium in a dish. The applicant found that too long sterilization in 75% alcohol may damage the quality of the rubber disk, leading to increased amount of scratches in the wounding bed. For repeated use, the stamp (still mounted on the shaft) is washed and primed with fresh culture medium. After wounding, culture surfaces with growing cells not contacted by the stamp should also be denuded of cells using an eyebrow brush. This step was found to be important because large numbers of cells were found by the applicant to be spared from wounding along the peripheral flange areas and lateral walls of the dish. Furthermore, all wounded dishes should be checked visually against a black background and microscopically to identify unwounded areas or cell bands that are unacceptably wide. These areas are then manually scraped with a brush to remove the undesired cells. If the acceptable areas are less than 60% of the total, the dish should be rejected. The remaining wounded cells are rinsed gently with culture medium several times and returned to the incubator. After use, stamps are rinsed clean with strong streams of de-ionized water.

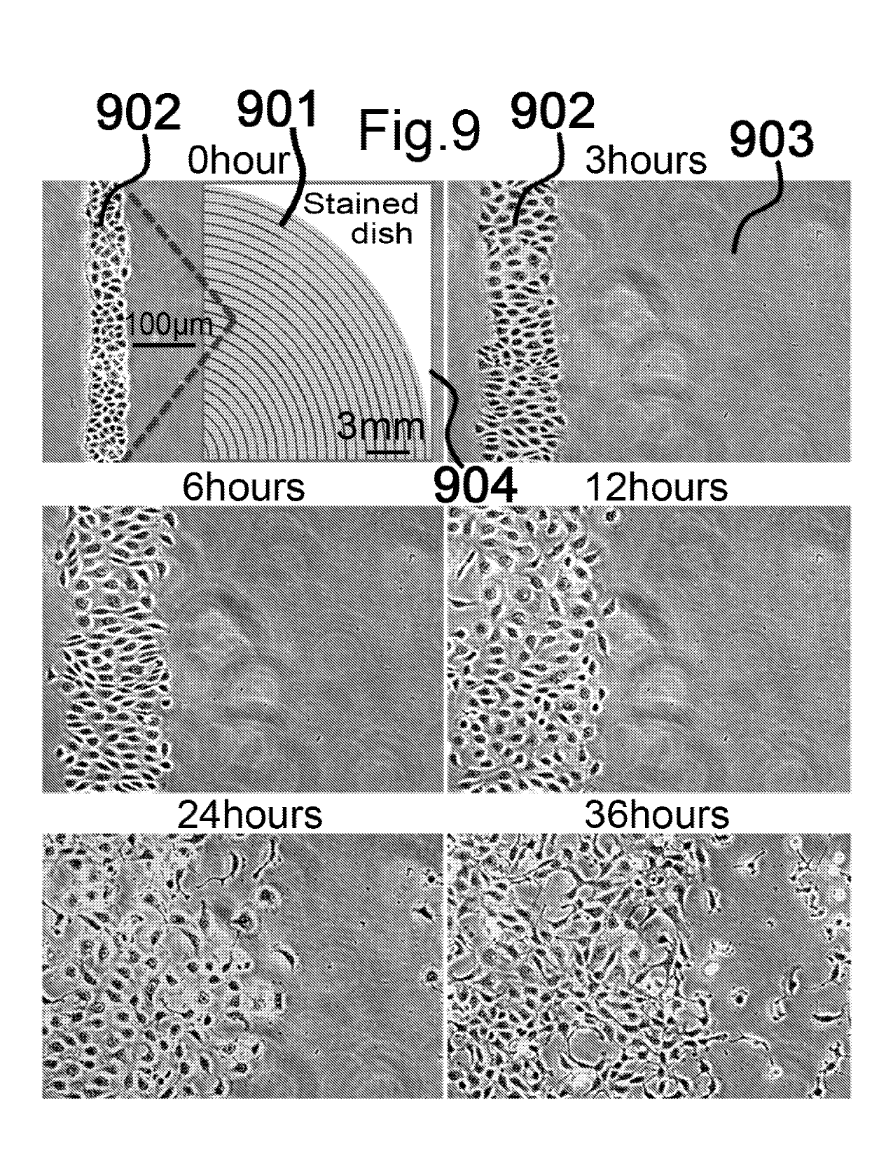

In one application of present invention, a cell monolayer in a culture dish was wounded with concentric stamp assembly mounted on pneumatically driven device, and then morphological observations were performed by time-lapse video microscopy. The diagram representing cell monolayer wounded in a concentric circular pattern, part of a stained dish 904 and the still frames of part of a cell strip 902 up to 24 hours by time-lapse video microscopy are shown in FIG. 9. The wounding bed 903 is clear of scratches after wounding. The cells in the strip migrate out to the wounding bed, separate from each other, and proliferate into more cells. The cell strip 902 corresponds to part of a strip 901 in a stained dish 904.

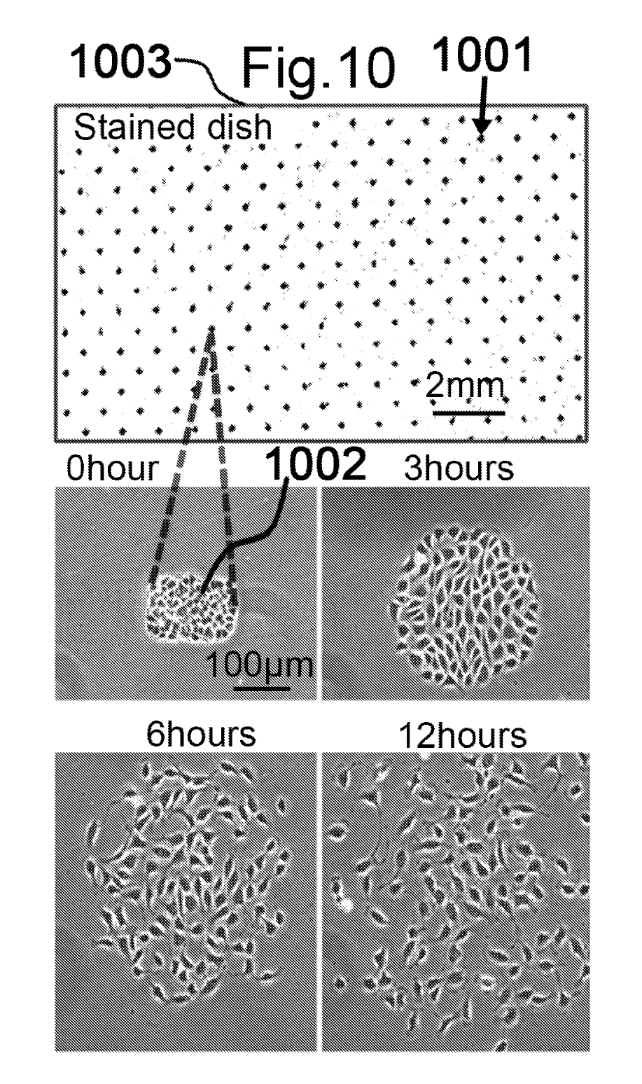

In another application of present invention, a cell monolayer in a culture dish was wounded with concentric stamp assembly mounted on pneumatically driven device, and then morphological observations were performed by time-lapse video microscopy. The diagram representing part of a stained dish 1003 and the still frames of a cell island 1002 produced by two wounding operations at right angle with a parallel stamp by time-lapse video microscopy are shown in FIG. 10. Cells in the island 1005 separate from each other much more completely and earlier than the cells in cells in a strip 902. The cell island 1002 corresponds to a small dot 1001 in a stained dish 1003.

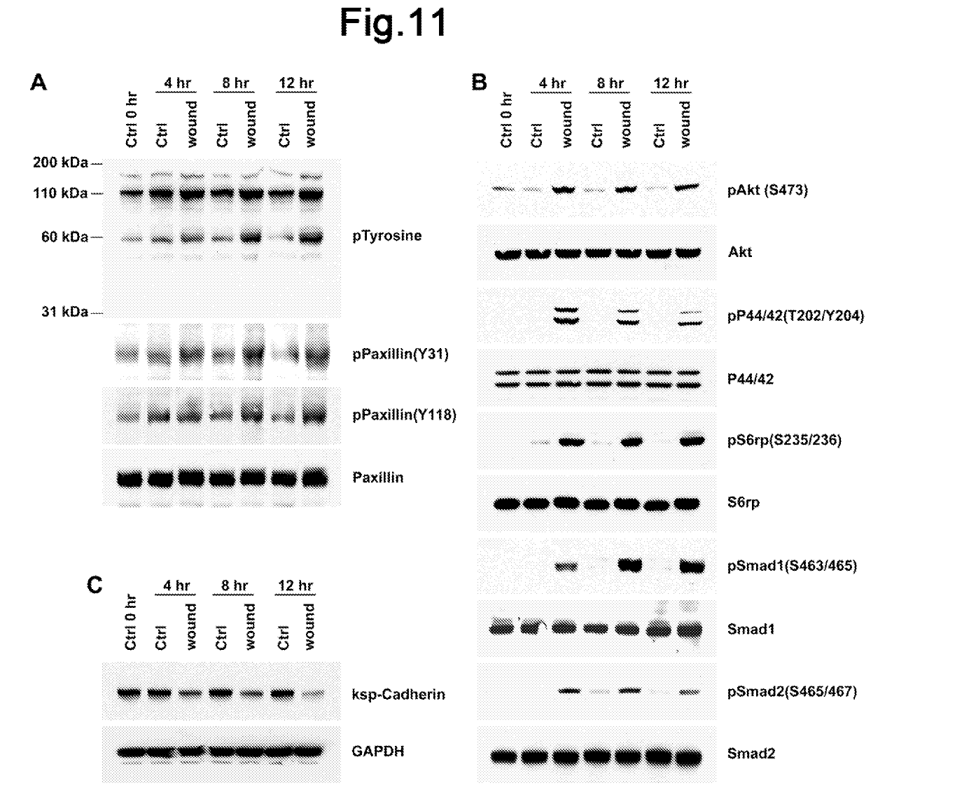

In another application of present invention, multiple dishes containing cell monolayers were wounded with concentric stamp assembly mounted on pneumatically driven device, and then samples were harvested on remaining cells at different time points after wounding, which were subjected to Western Blot Protein Analysis on multiple target proteins. FIG. 11 represents the pictures of Western Blots of proteins in SDS extracts of control cells and wounded cells after 4, 8 and 12 hours, which show activation and phosphorylation of multiple proteins in several signaling pathways.

Although preferred embodiments of the invention are described in this document, some further modifications or variations can be easily made following the general principles of present invention. It will be understood that the scope of the invention would intend to cover any apparent modifications and variations if judged by those with skill in the art to which the invention pertains to be included in what such claims are entitled.

What is claimed is:

1. A device of a stamp assembly which is applied to a cultured cell monolayer, comprising: a stamp disk engraved with alternating concentric circular grooves and ridges, further engraved with radial grooves; a stamp support block for the stamp disk connecting to the stamp disk.

2. The device of claim 1, further comprising: wherein said support block of said stamp assembly comprises two parts; and wherein when the first part not directly attaching to the stamp disk is held at a position, said stamp assembly is adjusted to control the extension of the second part of said stamp assembly which is directly attaching to the stamp disk from the position of first part, while allowing the rotation of said stamp disk to dislodge cultured cell monolayer.

3. A device of a stamp assembly which is applied to a cultured cell monolayer, comprising: a stamp disk engraved with alternating parallel grooves and ridges; wherein a stamp support block holds and moves the stamp disk along the axis of the parallel ridges on the stamp disk.

4. A system of in-vitro wound healing model, comprising: a main stand; a stamp assembly comprising a stamp disk and a support block for the stamp disk; wherein the stamp assembly is connected to a shaft of a main stand; wherein a holding device holds a cell culture container containing cell culture monolayer under the stamp assembly; wherein the stamp assembly is moved to contact with and dislodge cultured cells of the cultured cell monolayer in the cell culture container; cell strips or islands are left behind after dislodging; morphological or biochemical studies are performed on remaining cell strips or islands.

5. The system of claim 4, further comprising: wherein said shaft is driven by a pneumatic actuator; wherein said shaft is controlled by a button to move said stamp assembly; wherein said pressure of the pneumatic actuator is adjusted by a pneumatic pressure regulator.

6. The system of claim 4, further comprising: wherein said shaft is driven by an electrical actuator; wherein said shaft is controlled by a button to move said stamp assembly; wherein said pressure of the electrical actuator is adjusted by the electrical pressure regulator.

7. The system of claim 4, further comprising: wherein said stamp disk engraved with alternating circular concentric grooves and ridges, further engraved with radial grooves; and wherein said stamp assembly moves circularly to dislodge cells on said cultured cell monolayer, and leave behind concentric circular cell bands alternating with wounding bed.

8. The system of claim 4, further comprising: wherein said stamp disk engraved with alternating parallel grooves and ridges; and wherein said stamp block moves stamp disk along the axis of the parallel bands, to dislodge cells on said cultured cell monolayer, and leave behind parallel cell bands alternating with wounding bed.

9. The system of claim 4, further comprising: wherein said stamp disk engraved with alternating parallel grooves and ridges; and wherein said stamp block moves stamp disk along the axis of the parallel bands, to dislodge cells on said cultured cell monolayer, and leave behind parallel cell bands alternating with wounding bed, and then the stamp assembly is lifted from said cultured cell monolayer and turned to a right angle to the first wounding direction, and then a second wounding on same said cultured cell monolayer would further generate cell islands.

10. The system of claim 4, further comprising: wherein said holding device holds a cell culture plate containing multiple culture wells, and wherein said stamp assembly is a stamp array composed of multiple small stamps, and wherein the small stamps are linked and rotated together.
